(19) United States
(12) Patent Application Publication
(10) Pub. No.: US 2024/0124767 A1
(43) Pub. Date: Apr. 18, 2024

(54) LIGHT-EMITTING COMPOSITION

(71) Applicant: Sumitomo Chemical Co., Ltd., Tokyo (JP)

(72) Inventors: Jonathan Behrendt, Cambridgeshire (GB); Kiran Kamtekar, Cambridgeshire (GB); Nazrul Islam, Cambridgeshire (GB)

(73) Assignee: Sumitomo Chemical Co., Ltd., Tokyo (JP)

(21) Appl. No.: 17/767,415

(22) PCT Filed: Oct. 8, 2020
(86) PCT No.: PCT/GB2020/052497

§ 371 Date: Apr. 7, 2022

(30) Foreign Application Priority Data
Oct. 8, 2019 (GB) 1914494.8

(51) Int. Cl. C09K 11/06; C09K 11/02; G01N 15/1434

(52) U.S. Cl. CPC C09K 11/06; C09K 11/02; G01N 15/1434; C09K 2211/1483; C09K 2211/1491; C09K 2211/185

(57) ABSTRACT

A light-emitting composition containing first and second light-emitting markers. The first light-emitting marker comprises a first light-emitting material and a first binding group configured to bind to a first target analyte. The second light-emitting marker comprises a second light-emitting material which is different from the first light-emitting material and a second binding group which is different from the first binding group and which is configured to bind to a second target analyte. A luminescent lifetime of the first light-emitting material is shorter than a luminescent lifetime of the second light-emitting material. The difference in lifetimes of the first and second light-emitting materials may be used to distinguish between the first and second target analytes in a sample, e.g. by time-gated flow cytometry.

### BACKGROUND

Embodiments of the present disclosure relate to light-emitting compositions. Embodiments of the present disclosure further relate to methods of preparing said light-emitting compositions and assays using said compositions.

Nanoparticles of silica and a light-emitting material have been disclosed as labelling or detection reagents.

Nanoscale Res. Lett., 2011, vol. 6, p 328 discloses entrapment of a small molecule in a silica matrix.

J. Mater. Chem., 2013, vol. 1, pp 3297-3304, Behrendt et al. describes silica-LEP nanoparticles.

WO 2018/060722 discloses composite particles comprising a mixture of silica and a light-emitting polymer having polar groups.

EP 2482072 discloses long lifetime photoluminescent probes that contain no metal complexes having a long lifetime luminescence energy donor and a fluorescence energy acceptor.

US 2015/0154184 discloses time-gated fluorescence imaging with silicon-containing particles.

Jiang et al, “Long-Lived Phosphorescent Iridium(III) Complexes Conjugated with Cationic Polyfluorenes for Heparin Sensing and Cellular Imaging”, Macromolecular

Rapid Communications, Vol. 37, Issue 7, p640-646 discloses time-gated luminescent imaging using polyelectrolytes containing long-lived phosphorescent Ir(III) complexes to eliminate short lived background fluorescence.

Shi et al, “Cationic Polyfluorenes with Phosphorescent Iridium(III) Complexes for Time-Resolved Luminescent Biosensing and Fluorescence Lifetime Imaging”

Advanced Functional Materials, Vol. 23, Issue 26, 3268-3276 discloses a time-resolved photoluminescence technique and fluorescence lifetime imaging microscopy for biosensing and bioimaging based on phosphorescent conjugated polyelectrolytes (PCPEs) containing Ir(III) complexes and polyfluorene units.

Jin et al, “Time-gated real-time bioimaging system using multicolor microsecond-lifetime silica nanoparticles” Proc. SPIE 7568, Imaging, Manipulation, and Analysis of Biomolecules, Cells, and Tissues VIII, 756819, February 24, 2010 discloses real-time background-free imaging and rare-event counting of microsecond-lifetime multi-colour labelled water-borne pathogens.

D. Ullmann, Comprehensive Medicinal Chemistry II, 2007, 3.28.7 “Lifetime Techniques Applied to Confocality Future Developments for High-Throughput Screening Read-Outs” discloses that the applicability of fluorescence lifetime methods to biological liquids is limited since changes in the fluorescence lifetime due to environmental quenching or polarity effects are not always predictable.

### SUMMARY

In some embodiments, the present disclosure provides a light-emitting composition comprising a first light-emitting marker and a second light-emitting marker. The first light-emitting marker contains a first light-emitting material and a first binding group configured to bind to a first target analyte. The second light-emitting marker has a second light-emitting material which is different from the first light-emitting material and a second binding group which is different from the first binding group and which is configured to bind to a second target analyte.

A luminescent lifetime of the first light-emitting material is shorter than a luminescent lifetime of the second light-emitting material.

Optionally, the luminescent lifetime of the first light-emitting material is at least 10 times shorter than that of the second light-emitting material.

Optionally, the first light-emitting material is selected from a fluorescent organic light-emitting material which does not comprise a metal complex and a fluorescent organic light-emitting material which comprises an aluminium metal complex. Optionally, the first light-emitting material is a first light-emitting polymer. Optionally, the first light-emitting polymer is a conjugated light-emitting polymer.

Optionally, the second light-emitting material is selected from an organic light-emitting material comprising a d-block or f-block metal complex. Optionally, the d-block or f-block metal complex is provided in an end-group, a repeat unit side-group or a repeat unit main chain group of a second light-emitting polymer. Optionally, the second light-emitting material is a phosphorescent iridium or platinum complex.

Optionally, at least one of the first and second light-emitting markers is a light-emitting particle having a light-emitting particle core containing the first or second light-emitting material and the first or second binding group bound to the light-emitting particle core.

Optionally, the light-emitting particle core further has a matrix material.

Optionally, at least one of the first and second light-emitting markers is dissolved in a solvent of the composition.

Optionally, at least one of the first and second binding groups is a biomolecule.

Optionally, the composition contains one or more further light-emitting markers.

According to some embodiments of the present disclosure, there is provided an assay method in which a sample is contacted with a light-emitting composition as described herein. The resulting mixture is irradiated and luminance of any of the light-emitting markers bound to the respective target analytes is measured after at least one predetermined time following the irradiation.

Optionally, the first and second light-emitting materials both absorb light of a single peak wavelength of the irradiation light.

Optionally the first and second light-emitting materials absorb, and are irradiated with, light having different peak wavelengths.

Optionally, the sample contacted with the light-emitting composition is analysed by flow cytometry.

Optionally, an amount of the first target analyte and the second target analyte bound to the respective first and second light-emitting markers is determined.

Optionally, the sample comprises a mixture of cells and one or more different types of target cells bound to the light-emitting marker are identified and/or quantified.

### DESCRIPTION OF THE DRAWINGS

The disclosed technology and accompanying figures describe some implementations of the disclosed technology.

FIG. 1 is a schematic illustration of a light-emitting composition according to some embodiments containing first and second light-emitting marker particles having different emission lifetimes;

FIG. 2 is a schematic illustration of the light-emitting composition of FIG. 1 mixed with a sample to be analysed;

FIG. 3 shows the photoluminescence spectra for a red fluorescent marker particle and a red phosphorescent marker particle; and

FIG. 4 is a graph luminance vs. time for the red fluorescent marker particle and red phosphorescent marker particle of FIG. 3 at emission in the range of 570-600 nm.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, electromagnetic, or a combination thereof. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily just the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while some aspect of the technology may be recited as a computer-readable medium claim, other aspects may likewise be embodied as a computer-readable medium claim, or in other forms, such as being embodied in a means-plus-function claim.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that different luminescent markers having different luminescent lifetimes can be distinguished from each other by measuring luminescence of the markers after a time delay following excitation of the markers, and optionally at different times following excitation of the luminescent materials. Two or more luminescent markers with different lifetimes may be configured to bind to different materials. In this way, a multiplex assay may be performed in which the presence and/or concentration of different target substances in the same sample can be identified even if two or more of the different luminescent materials have a similar peak emission wavelength, or to distinguish between luminescent materials having different peak emission wavelengths but overlapping emission peaks.

Optionally, and in addition to use of two or more luminescent materials with different luminescent lifetimes, the two or more different luminescent materials in a composition as described herein may be distinguished from one another by one or more of: use of two or more luminescent materials having different absorption peaks wherein a sample which has been contacted with the composition containing these luminescent materials is irradiated with two or more different wavelengths of light corresponding to the absorption peaks for the two or more different luminescent materials; and two or more different colours of emission for at least two of the two or more luminescent materials.

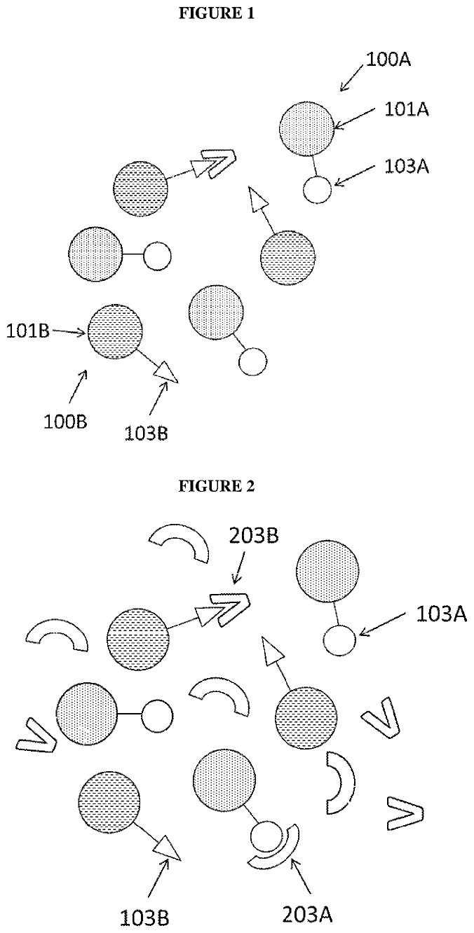

FIG. 1 illustrates a composition according to some embodiments in which the composition is in the form of luminescent particles.

The composition contains a plurality of first luminescent markers 100A and a plurality of second luminescent markers 100B. Each luminescent particle comprises or consists of a core containing a light-emitting material. The cores 101A of first luminescent particles 100A contain first light-emitting material. The cores 101B of second luminescent particles 100B contain a second light-emitting material.

In the embodiment of FIG. 1, the first and second luminescent markers are luminescent particles. However, in other embodiments, the first and/or second luminescent markers may be provided in dissolved form in a liquid of the composition and/or when in use. The composition may contain a liquid in which each of the light-emitting markers is independently in particulate form and is dispersed in the liquid or is dissolved in the liquid.

The first light-emitting material has a shorter luminescent lifetime than the second light-emitting material. By “luminescent lifetime” as used herein is meant the time taken for luminescent intensity to decay to 1/e of a starting luminescent intensity.

The first and second luminescent particles 100A, 100B may carry first and second binding groups 103A, 103B respectively wherein the binding groups 103A and 103B are configured to bind to different materials, e.g. different biomolecules. First and second binding groups 103A, 103B may be bound, e.g. covalently bound, to respective cores 101A and 101B.

With reference to FIG. 2, in use the composition containing the luminescent particles may be brought into contact with a sample to be analysed containing target analytes 203A and 203B.

The first binding group 103A of the first light-emitting particle 100A may selectively bind to a first target analyte 203A. The second binding group 103B of the second light-emitting particle 100B may selectively bind to a second target analyte 203B.

Particles bound to a target analyte may be differentiated from unbound particles using any technique known to the skilled person including, without limitation, separating particles bound to a target analyte from particles which are not so bound, e.g. a plate assay in which target analytes bind to a surface allowing for light-emitting particles not bound to a target analyte to be washed off the surface; and flow cytometry.

The particles are irradiated with one or more wavelengths of light, optionally irradiation by one or more lasers. Particles may be irradiated individually, e.g. as in flow cytometry, or a plurality of particles, e.g. all particles in the composition, may be irradiated simultaneously. A composition as described herein may be analysed following contact with a sample by time-gated flow cytometry, for example using flow cytometers as disclosed in Houston et al, “Overview of Fluorescence Lifetime Measurements in Flow Cytometry”, Methods Mol Biol. 2018;1678:421-446. doi: 10.1007/978-1-4939-7346-0_18.

In some embodiments, following irradiation, luminance of the irradiated particle or particles is measured at times T1 and T2. T1 may be 0 or a non-zero value. T2 is greater than T1. The rate of decay of luminance of the first and second light-emitting materials between T1 and T2 is known, and is different for the first and second light-emitting materials. The cumulative measured luminance of the first and second light-emitting materials at times T1 and T2, combined with the known rate of decay of the luminance of the first and second light-emitting materials, allows for the cumulative measurements to be resolved into a luminescence measurement for each of the first and second light-emitting materials.

In some embodiments, luminance is measured after a time T3 wherein T3 is a non-zero value after luminance from the first light-emitting material has fallen to at least 1/100 or 1/1000 of its starting luminance but luminance from the second light-emitting material has fallen to less than 1/10 of its starting luminance. Any luminance present at this time T3 may be attributed to the second light-emitting material. An earlier measurement at a time before luminance of the first light-emitting material has fallen to 1/100 of its starting luminance may, in combination with the measurement at T3, be used to resolve the measurements into measurements for the first and second light-emitting materials specifically.

Measuring luminescence from the light-emitting materials at different times, which is referred to hereinafter as time-resolved luminescence measurement, allows for luminance from the first and second light-emitting materials to be distinguished from one another based on the known rate of decay of luminance of the first and second light-emitting materials.

Accordingly, distinguishing between the first and second light-emitting markers includes one or more of: measuring a change in luminance between two time points; and measuring luminance at a time point after luminance of the first light-emitting material, but not the second light-emitting material, has fallen to 0.

One or more other parameters may be used to distinguish between the first and second light-emitting particles, including the peak wavelengths of the particles in the case where these peaks are distinguishable from one another, e.g. are at least 10 nm apart.

In some embodiments, the first light-emitting material and the second light-emitting material have similar peak emission wavelengths, optionally peak emission wavelengths that are less than 50 nm from each other, optionally within 20 nm or within 10 nm of each other.

In some embodiments, the first light-emitting material and the second light-emitting material have peak emission wavelengths that are at least 50 nm apart from one another, optionally at least 100 nm apart.

In some embodiments, the first and second light-emitting materials absorb light of the same excitation wavelength and the first and second light-emitting materials are irradiated with light of the excitation wavelength, and optionally one or more further wavelengths.

In some embodiments, the first and second light-emitting materials absorb light at different excitation wavelengths and the first and second light-emitting materials are irradiated with light of the at least two different excitation wavelengths, and optionally one or more further wavelengths.

It will be understood that luminescence of markers upon irradiation as described herein is photoluminescence. Optionally, the markers as described herein are not chemiluminescent.

FIGS. 1 and 2 illustrate light-emitting particles having a binding group bound to a light-emitting particle which may be dispersed in the sample when in use. In other embodiments, the binding group may be bound to a light-emitting material which is in dissolved form when in use.

FIGS. 1 and 2 illustrate a composition containing two different light-emitting markers. In other embodiments, the composition may contain three or more different light-emitting markers wherein at least two of the light-emitting materials have different luminescent lifetimes.

### First and Second Light-Emitting Materials

The first and second light-emitting materials have different luminescent lifetimes.

In some embodiments, the luminescent lifetime of the second light-emitting material may be at least 10 or at least 100 times greater than that of the first light-emitting material.

Luminescent lifetimes of various light-emitting materials are given in Chem. Rev.2010, 110 (5), 2641-2684, the contents of which are incorporated herein by reference.

Each light-emitting material may emit light having a peak wavelength in the range of 350-1000 nm.

A blue light-emitting material as described herein may have a photoluminescence spectrum with a peak of no more than 500 nm, preferably in the range of 400-500 nm, optionally 400-490 nm.

A green light-emitting material as described herein may have a photoluminescence spectrum with a peak of more than 500 nm up to 580 nm, optionally more than 500 nm up to 540 nm.

A red light-emitting material as described herein may have a photoluminescence spectrum with a peak of no more than 580 nm up to 950 nm, optionally up to 630 nm, optionally 585 nm up to 625 nm.

The light-emitting material may have a Stokes shift in the range of 10-850 nm.

UV/vis absorption spectra of light-emitting materials as described herein may be as measured in methanol solution or suspension using a Cary 5000 UV-vis-IR spectrometer.

Photoluminescence spectra of light-emitting materials as described herein may be measured in methanol solution or suspension using a Jobin Yvon Horiba Fluoromax-3.

The first light-emitting material and the second light-emitting material may emit light from different excited states. Emission may be selected from two or more of:
prompt fluorescence by decay of a singlet exciton from a lowest singlet excited state S¹ to a ground state S⁰;
phosphorescence by decay of a triplet exciton from the lowest triplet excited state T¹ to a ground state S⁰;
emission from a ⁵ D excited state, e.g. decay of a lanthanide exciton from a ⁵ D excited state to a ⁷ F ground state; and
thermally activated delayed fluorescence (TADF), as distinct from prompt fluorescence, in which the excited state of a light-emitting material changes to a higher energy level, e.g. T₁ to S₁, followed by fluorescence by decay from the higher energy level.

Each light-emitting material may be selected from inorganic and organic light-emitting materials known to the skilled person.

Inorganic light-emitting materials include, without limitation, quantum dots.

Organic light-emitting materials include non-polymeric light-emitting materials and light-emitting polymers.

An organic light-emitting material as described herein may comprise or consist of a metal complex. Exemplary metal complexes include fluorescent p-block metal complexes, e.g. tris-(8-hydroxyquinoline)aluminum (Alq3); phosphorescent d-block metal complexes, e.g. phosphorescent complexes of Rhenium, Osmium, Iridium, Platinum or Gold, preferably Iridium or Platinum; and luminescent lanthanide complexes, e.g. complexes of Europium or Terbium.

Exemplary phosphorescent iridium complexes include:

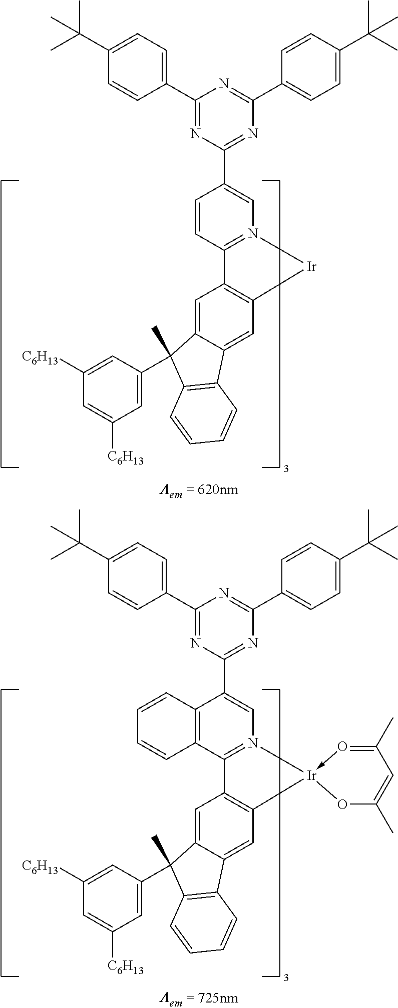

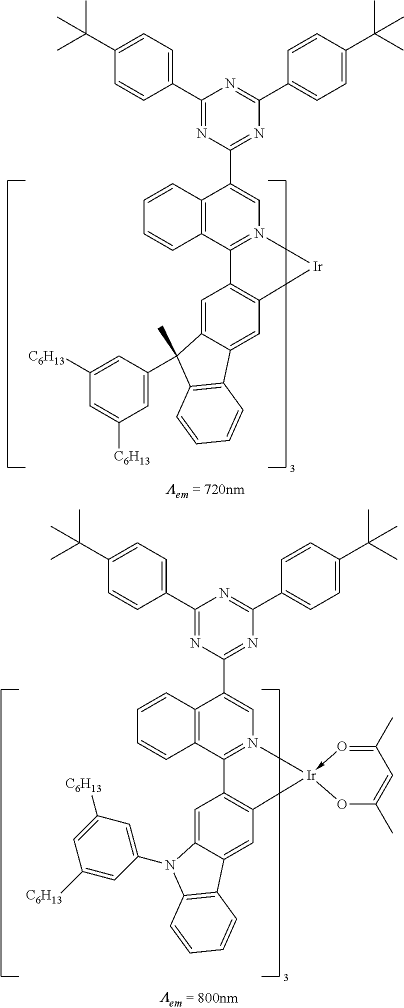

Exemplary phosphorescent platinum complexes include:

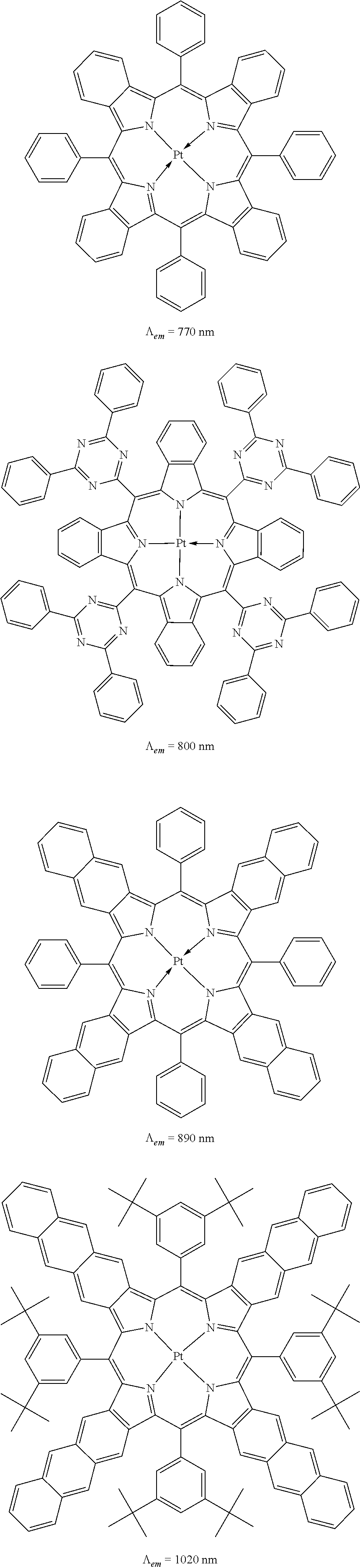

A light-emitting metal complex as described herein, e.g. a d-block phosphorescent metal complex or a Europium complex, may be provided as an end group, repeat unit side group or repeat unit main chain group of a light-emitting polymer. The light-emitting polymer may have a conjugated or non-conjugated polymer backbone.

Exemplary non-polymeric fluorescent materials include, without limitation: fluorescein; fluorescein isothiocyanate (FITC); fluorescein NHS; Alexa Fluor 488; Dylight 488; Oregon green, DAF-FM; 6-FAM; 2,7-dichlorofluorescein; 3′-(p-aminophenyl)fluorescein; 3′-(hydroxyphenyl)fluorescein; rhodamines, for example Rhodamine 6G and Rhodamine 110 chloride; coumarins; boron-dipyrromethenes (BODIPYs); naphthalimides; perylenes; benzanthrones; benzoxanthrones;

benzothiooxanthrones; 2-(4-pyridyl)-5-phenyl-oxazole; 2-quinolinyl-5-phenyl-oxazole; 2-(4-pyridyl)-5-naphthyl-oxazole; 2-(4-pyridyl)-5-phenyl-thiazole; 2-quinolinyl-5-phenyl-thiazole; 2-(4-pyridyl)-5-naphthyl-thiazole; 2-(4-pyridyl)-5-phenyl-thiophene; 2-quinolinyl-5-phenyl- thiophene; 2-(4-pyridyl)- 5-naphthyl- thiophene and salts thereof, each of which may be unsubstituted or substituted with one or more substituents. Exemplary substituents are ionic or non-ionic substituents as described herein, optionally chlorine, alkyl amino; phenylamino; and hydroxyphenyl.

In some embodiments, a non-polymeric light-emitting material as described herein is mixed with a polymeric or non-polymeric host material. In the case of a light-emitting particle, the particle may comprise a mixture of the light-emitting material and the host. In the case where the light-emitting material is dissolved, the light-emitting material and host material may both be bound directly or indirectly to a common unit of the binding group. Optionally, the light-emitting material and host are each bound to a linking molecule and the linking molecules are bound to the binding group. Optionally, each linking molecule is selected from streptavidin, neutravidin, avidin or a recombinant variant or derivative thereof and the binding group comprises or consists of biotin which the linking molecules are bound to.

Light-emitting polymers are preferred.

The light-emitting polymer may be a homopolymer or may be a copolymer comprising two or more different repeat units.

The light-emitting polymer may have a non-conjugated backbone or may be a conjugated polymer. By “conjugated polymer” is meant a polymer comprising repeat units in the polymer backbone that are directly conjugated to adjacent repeat units. Conjugated light-emitting polymers include, without limitation, polymers comprising one or more of arylene, heteroarylene and vinylene groups conjugated to one another along the polymer backbone.

The light-emitting polymer may have a linear, branched or crosslinked backbone.

The light-emitting polymer may have a solubility of at least 0.01 mg/ml in an alcoholic solvent, optionally in the range of 0.01-10 mg/ml. Optionally, solubility is at least 0.1 or 1 mg/ml. The solubility is measured at 25° C. Preferably, the alcoholic solvent is a C₁₋₁₀ alcohol, more preferably methanol. Solubility of the light-emitting polymer may be adjusted by selection of substituents of the polymer.

Preferably, a light-emitting polymer as described herein contains at least one arylene repeat unit, optionally one or more arylene repeat units selected from phenylene, fluorene, benzofluorene, phenanthrene, dihydrophenanthrene, naphthalene and anthracene, more preferably fluorene or phenylene, most preferably fluorene. The, or each, arylene repeat unit may be unsubstituted or substituted with one or more substituents, preferably one or more substituents selected from non-polar and polar substituents as described above. The repeat units and/or extent of conjugation of a light-emitting polymer may be selected to tune its absorption peak.

Arylene co-repeat units may be selected from repeat units of formulae (III)-(VI):

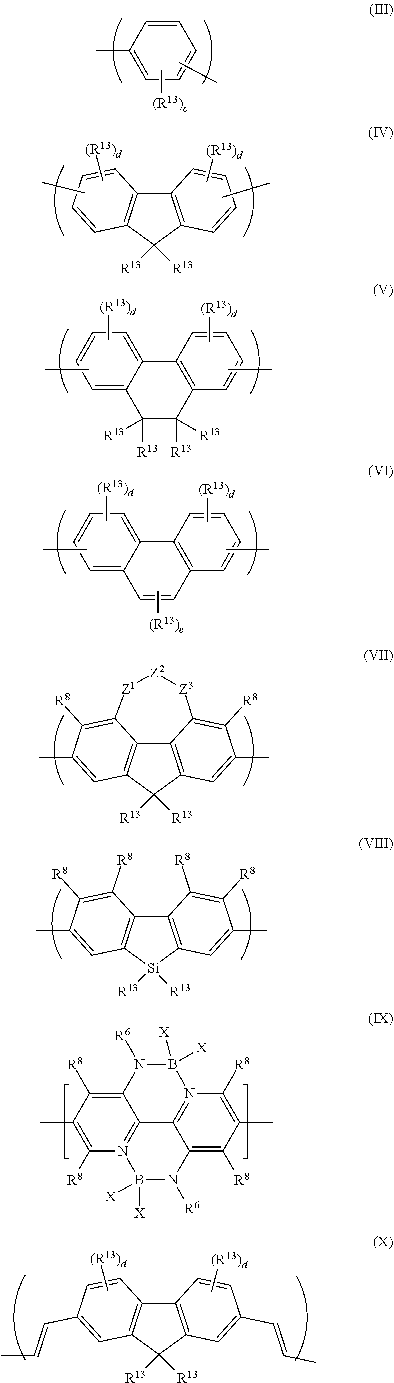

wherein:

R⁸ in each occurrence is independently H or R¹³, preferably H;

R⁶ is a C₁₋₁₂ hydrocarbyl group, optionally a C₁₋₁₂ alkyl group or C₁₋₄ alkyl group;

X independently in each occurrence is a substituent, preferably a substituent selected from the group consisting of branched, linear or cyclic C₁₋₂₀ alkyl; phenyl which is unsubstituted or substituted with one or more substituents, e.g. one or more C₁₋₁₂ alkyl groups; and F;

Z¹ -Z²-Z³ is a C₂ (ethylene) chain or C₃ alkylene (propylene) chain wherein one or two non-adjacent C atoms may be replaced with O, S or NR⁶;

R¹³ in each occurrence is independently a substituent;

c is 0, 1, 2, 3 or 4, preferably 1 or 2;

each d is independently 0, 1, 2 or 3, preferably 0 or 1;

and e is 0, 1 or 2, preferably 2.

R¹³ may be selected from ionic and non-ionic groups.

Exemplary non-ionic groups are:
C₁₋₂₀ alkyl wherein one or more non-adjacent, non-terminal C atoms may be replaced with O, S, CO, COO, NR¹ or SiR¹₂ wherein R¹ is a C₁₋₁₂ hydrocarbyl group; and
C₆₋₂₀ aryl, e.g. phenyl, which is unsubstituted or substituted with one or more substituents, optionally one or more substituents selected from F; CN; NO₂; and

C₁₋₂₀ alkyl, optionally C₁₋₁₂ alkyl wherein or more non-adjacent, non-terminal C atoms may be replaced with O, S, CO, COO, NR¹ or SiR¹₂.

A “non-terminal C atom” of an alkyl group as used herein means a C atom other than the C atom of the methyl group at the end of an n-alkyl group or the C atom of the methyl groups at the ends of a branched alkyl chain.

A preferred non-ionic group is formula —(Ar¹)ₚ—[O(R³O)ₜ-R⁴]_{q} wherein Ar¹ is an arylene group, preferably phenylene; p is 0 or 1; q is 1 if p is 0; q is at least 1, optionally 1, 2 or 3, if q is 1; R³ in each occurrence is a C₁₋₁₀ alkylene group, optionally a C₁₋₅ alkylene group, preferably ethylene; R⁴ is H or C₁₋₅ alkyl, and t is at least 1, optionally 1-20. Preferably, t is at least 2. More preferably, t is 2 to 5. The value oft may be the same in all the polar groups of formula —O(R³O) -R⁴ . The value of t may differ between polar groups of the same polymer.

C₁₋₁₂ hydrocarbyl groups as described herein include, without limitation, C₁₋₁₂ alkyl, unsubstituted phenyl and phenyl substituted with one or more C₁₋₆ alkyl groups.

Exemplary ionic groups have formula —(Sp)m-(R²)n wherein Sp is a spacer group; m is 0 or 1; R² independently in each occurrence is an ionic group; n is 1 if m is 0; and n is at least 1, optionally 1, 2, 3 or 4, if m is 1.

Preferably, Sp is selected from:
C₁₋₂₀ alkylene or phenylene-C₁₋₂₀ alkylene wherein one or more non-adjacent C atoms may be replaced with O, S, N or C═O;
a C₆₋₂₀ arylene or 5-20 membered heteroarylene, more preferably phenylene, which, in addition to the one or more substituents R² may be unsubstituted or substituted with one or more substituents, optionally one or more C₁₋₂₀ alkyl groups wherein one or more non-adjacent, non-terminal C atoms may be replaced with O, S, CO, COO, NR¹ or SiR¹₂.

In a preferred embodiment, Sp is a C₆₋₂₀ arylene substituted, in addition to R² , with one or more groups of formula —[O(R³O)ₜ-R⁴]_{q} as described herein.

R² may be an anionic or cationic group, preferably anionic. Exemplary anionic groups are —COO⁻, a sulfonate group; hydroxide; sulfate; phosphate; phosphinate; or phosphonate. An exemplary cationic group is —N(R⁵)₃⁺ wherein R⁵ in each occurrence is H or C₁₋₁₂ hydrocarbyl. Preferably, each R⁵ is a C₁₋₁₂ hydrocarbyl.

If the conjugated organic material is substituted with one or more ionic substituents then the polymer further comprises a charge-balancing counterion to balance the charge of the ionic substituents.

Cationic counterions are optionally selected from a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

Anionic counterions are optionally selected from a halide; a sulfonate group, optionally mesylate or tosylate; hydroxide; carboxylate; sulfate; phosphate; phosphinate; phosphonate; or borate.

The repeat units of a light-emitting polymer may consist of one or more arylene repeat units as described herein.

A fluorescent light-emitting polymer may comprise one or more arylene repeat units as described herein and one or more co-repeat units, optionally one or more co-repeat units selected from heteroarylene repeat units and amine repeat units.

If co-repeat units are present then the arylene repeat units preferably form 50-99.5 mol % or 80-99.5 mol % of the repeat units of the polymer.

Exemplary heteroarylene co-repeat units include fused or unfused thiophene repeat units, e.g. thiophene, thienothiophene, dithienothiophene, benzodithiophene, benzothiadiazole and combinations thereof, each of which may be unsubstituted or substituted with one or more substituents, e.g substituents R¹³ as described above.

Heteroarylene co-repeat units are optionally selected from formulae (VII), (VIII) and (IX):

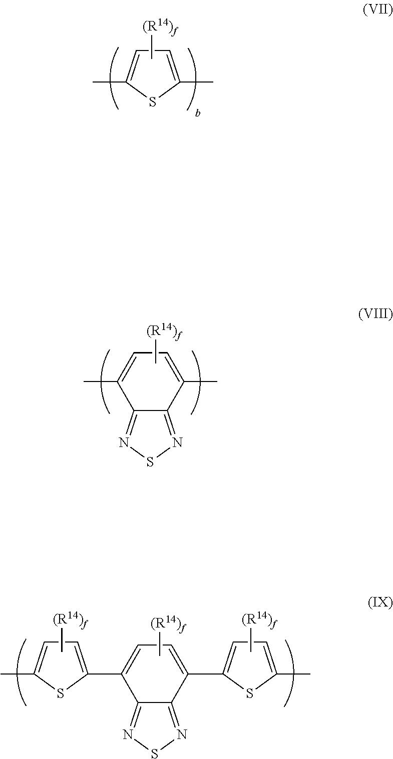

wherein R¹⁴ in each occurrence is H or a substituent R¹³ as described above; f is 0, 1 or 2; and, when f is 2, the two R¹⁴ groups may be linked to form an aromatic or non-aromatic ring.

Amine repeat units of a fluorescent light-emitting polymer may have formula (XII):

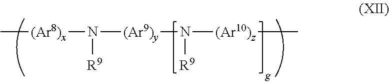

wherein Ar⁸, Ar⁹ and Ar¹⁰ in each occurrence are independently selected from substituted or unsubstituted aryl or heteroaryl, g is 0, 1 or 2, preferably 0 or 1, R⁹ independently in each occurrence is a substituent, and x, y and z are each independently 1,2 or 3.

R⁹, which may be the same or different in each occurrence when g is 1 or 2, is preferably selected from the group consisting of alkyl, optionally C₁₋₂₀ alkyl, Ar¹¹ and a branched or linear chain of Ar¹¹ groups wherein Ar¹¹ in each occurrence is independently substituted or unsubstituted aryl or heteroaryl.

Any two aromatic or heteroaromatic groups selected from Ar⁸, Ar⁹, and, if present, Ar m and Ar¹¹ that are directly bound to the same N atom may be linked by a direct bond or a divalent linking atom or group. Preferred divalent linking atoms and groups include O, S; substituted N; and substituted C.

Ar⁸ and Ar¹⁰ are preferably C₆₋₂₀ aryl, more preferably phenyl, that may be unsubstituted or substituted with one or more substituents.

In the case where g=0, Ar⁹ is preferably C₆₋₂₀ aryl, more preferably phenyl, that may be unsubstituted or substituted with one or more substituents.

In the case where g=1, Ar⁹ is preferably C₆₋₂₀ aryl, more preferably phenyl or a polycyclic aromatic group, for example naphthalene, perylene, anthracene or fluorene, that may be unsubstituted or substituted with one or more substituents.

R⁹ is preferably Ar¹¹ or a branched or linear chain of Ar¹¹ groups. Ar¹¹ in each occurrence is preferably phenyl that may be unsubstituted or substituted with one or more substituents.

Exemplary groups R⁹ include the following, each of which may be unsubstituted or substituted with one or more substituents, and wherein * represents a point of attachment to N:

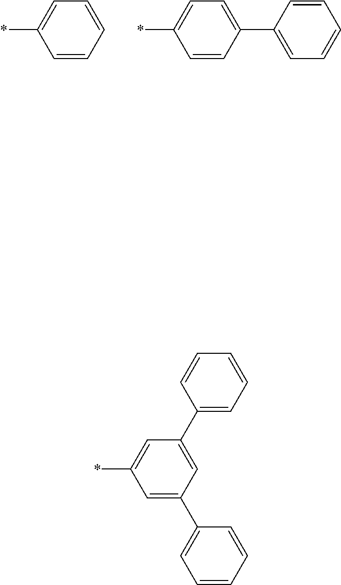

x, y and z are preferably each 1.

Ar⁸, Ar⁹, and, if present, Ar¹⁰ and Ar¹¹ are each independently unsubstituted or substituted with one or more, optionally 1, 2, 3 or 4, substituents.

Substituents may independently be a group R¹³ as described above.

Preferred substituents of Ar⁸, Ar⁹, and, if present, Ar¹⁰ and Ar¹¹ are C₁₋₄₀ hydrocarbyl, preferably C₁₋₂₀ alkyl.

Preferred repeat units of formula (XII) include unsubstituted or substituted units of formulae (XII-1), (XII-2) and (XII-3):

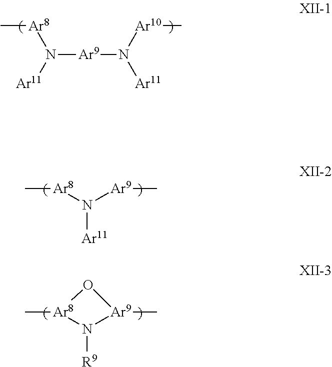

A light-emitting polymer may contain one or more arylene repeat units as described herein which may be unsubstituted or substituted with one or more polar or non-polar substituents as described herein and one or more of: an end-group comprising a light-emitting metal complex; a repeat unit having a side group comprising a light-emitting metal complex; and a light-emitting complex provided in a main chain of the light-emitting polymer.

The or each repeat unit of the polymer may be selected to produce a desired colour of emission of the polymer.

The polystyrene-equivalent number-average molecular weight (Mn) measured by gel permeation chromatography of the light-emitting polymers or the silica polymers described herein may be in the range of about 1×10³ to 1×10⁸, and preferably 1×10⁴ to 5×10⁶. The polystyrene-equivalent weight-average molecular weight (Mw) of the polymers described herein may be 1×10³ to 1×10⁸, and preferably 1×10⁴ to 1×10⁷.

Polymers as described herein are suitably amorphous polymers.

### Light-Emitting Particle

In the case where a light-emitting material as described herein (e.g. a first light-emitting material or a second light-emitting material) is provided in a core of a light-emitting particle, the particle core may comprise or consist of the light-emitting material and a matrix material. Matrix materials include, without limitation, inorganic matrix materials, optionally inorganic oxides, optionally silica. The matrix may at least partially isolate the light-emitting material from the surrounding environment. This may limit any effect that the external environment may have on the lifetime of the light-emitting material.

The light-emitting material may be mixed with the matrix material.

The light-emitting material may be bound, e.g. covalently bound, to the matrix material.

In some embodiments, the particle core may be formed by polymerisation of a silica monomer in the presence of a light-emitting material, for example as described in WO 2018/060722, the contents of which are incorporated herein by reference.

In some embodiments, the particle core comprises an inner core which comprises or consists of at least one light-emitting material and at least one shell surrounding the inner core. The at least one shell may be silica.

Optionally, at least 0.1 wt % of total weight of the particle core consists of the light-emitting material. Preferably at least 1, 10, 25 wt % of the total weight of the particle core consists of the light-emitting material.

Optionally at least 50 wt % of the total weight of the particle core consists of the silica. Preferably at least 60, 70, 80, 90, 95, 98, 99, 99.5, 99.9 wt % of the total weight of the particle core consists of silica.

The particle core as described herein is the light-emitting particle without any surface groups, e.g. binding groups or solubilising groups, thereon.

In one embodiment of the present disclosure, at least 70 wt % of the total weight of the particle core consists of the light-emitting material or materials and silica. Preferably at least 80, 90, 95, 98, 99, 99.5, 99.9 wt % of the total weight of the particle core consists of the light-emitting material and silica. More preferably the particle core consists essentially of the light-emitting material and silica.

Light-emitting particles as described herein may be provided as a colloidal suspension comprising the particles suspended in a liquid. Preferably, the liquid is selected from water, C₁₋₁₀ alcohols and mixtures thereof. Preferably, the particles form a uniform (non-aggregated) colloid in the liquid. In some embodiments, each of the first, second and any further light-emitting markers are light-emitting particles dispersed in the liquid. In some embodiments, one or more of the light-emitting markers is in particle form dispersed in the liquid and one or more of the light-emitting markers is dissolved in the liquid.

The liquid may be a solution comprising salts dissolved therein, optionally a buffer solution.

In some embodiments, the particles may be stored in a powder form, optionally in a lyophilised or frozen form.

### Binding Groups

The binding groups of the first and second light-emitting materials may be selected according to a target analyte of a sample to be analysed.

In the case of a light-emitting particle, the first and second binding groups may be bound to a surface of the respective first and second light-emitting particle cores, e.g. bound to a matrix material of the light-emitting particle cores. Each binding group may be directly bound to the surface of a light-emitting particle core or may be spaced apart therefrom by one or more surface binding groups. The surface binding group may comprise polar groups. Optionally, the surface binding group comprises a polyether chain. By “polyether chain” as used herein is meant a chain having two or more ether oxygen atoms.

The surface of a light-emitting particle core may be reacted to form a group at the surface capable of attaching to a binding group or a surface binding group. Optionally, a silica-containing particle is reacted with a siloxane.

The binding group may be a biomolecule binding group. Biomolecule binding groups may be selected from the group consisting of: DNA, RNA, peptides, carbohydrates, antibodies, antigens, enzymes, proteins and hormones. The biomolecule binding group may be selected according to a target biomolecule to be detected.

A preferred biomolecule binding group is biotin. In some embodiments, the biotin biomolecule binding group binds directly to a target analyte.

In some embodiments, the biotin biomolecule binding group is bound to a protein having a plurality of biotin binding sites, preferably streptavidin, neutravidin, avidin or a recombinant variant or derivative thereof and biotinylated biomolecule having a second biotin group is bound to the same protein. The biotinylated biomolecule may be selected according to the target analyte. The biotinylated biomolecule may comprise an antigen binding fragment, e.g. an antibody, which may be selected according to a target antigen.

### Applications

Light-emitting markers as described herein may be used as luminescent probes for detecting or labelling a biomolecule or a cell. In some embodiments, the particles may be used as a luminescent probe in an immunoassay such as a lateral flow or solid state immunoassay. Optionally the particles are for use in fluorescence microscopy, flow cytometry, next generation sequencing, in-vivo imaging, or any other application where a composition containing the first and second light-emitting markers configured to bind to respective first and second target analytes are brought into contact with a sample to be analysed. The analysis may be performed using time-resolved spectroscopy. The applications can medical, veterinary, agricultural or environmental applications whether involving patients (where applicable) or for research purposes.

In use, the binding group of the particles may bind to target biomolecules which include without limitation DNA, RNA, peptides, carbohydrates, antibodies, antigens, enzymes, proteins and hormones. It will be understood that the binding group may be selected according to the target biomolecule. The target biomolecule may or may not be a biomolecule, e.g. a protein, at a surface of a cell.

A sample to be analysed may brought into contact with the particles, for example the particles in a colloidal suspension.

In some embodiments, the sample following contact with the particles is analysed by flow cytometry. In flow cytometry, the particles are irradiated by at least one wavelength of light, optionally two or more different wavelengths, e.g. one or more wavelengths including at least one of 355, 405, 488, 530, 562 and 640 nm, each of which may be ±10 nm. Light emitted by the light-emitting markers may be collected by one or more detectors. Detectors may be selected from, without limitation, photomultiplier tubes and photodiodes. To provide a background signal for calculation of a staining index, measurement may be made of particles mixed with cells which do not bind to the particles.

In some embodiments, the target analytes may be immobilised on a surface which is brought into contact with the light-emitting markers.

### EXAMPLES

Fluorescent Light-Emitting Polymer 1 and Phosphorescent Light-Emitting Polymer 1 were prepared by Suzuki polymerisation as disclosed in WO 00/53656 of the monomers set out in Table 1, followed by conversion of the COOEt group of Monomer Example 1 to a COO-Cs+ group as described in WO 2012/133229.

| | Fluorescent Light-Emitting | Phosphorescent Light- |
|---|---|---|
| Monomer | Polymer 1 (mol %) | Emitting Polymer 1 |
| 1 | 95 | 94 |
| 2 | 5 | — |
| 3 | — | 6 |

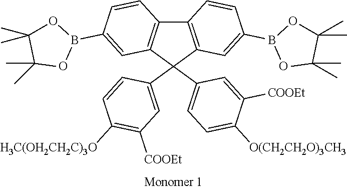

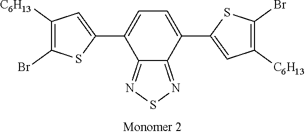

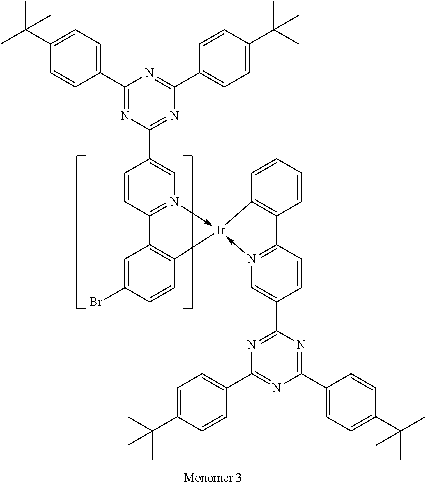

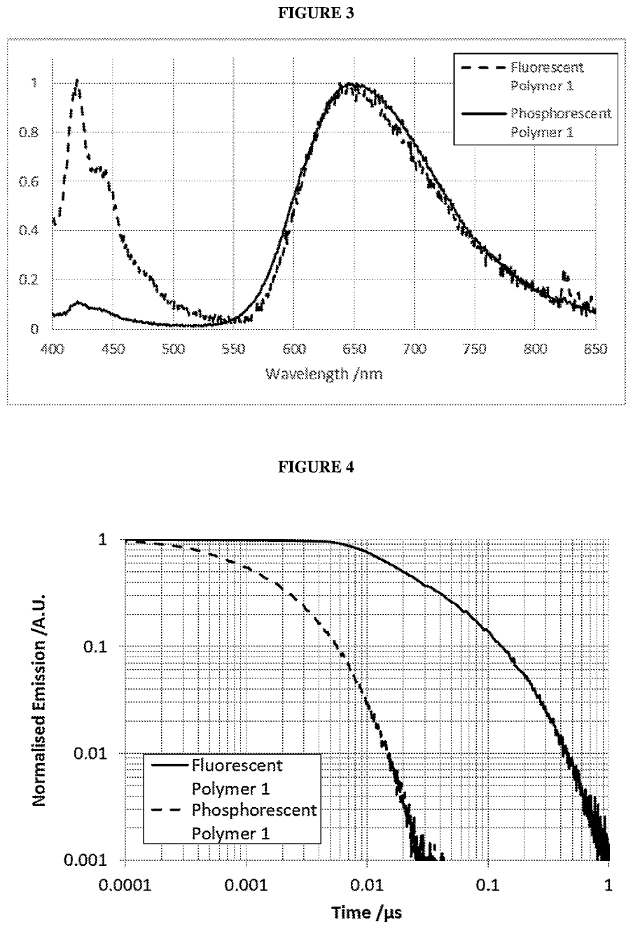

With reference to FIG. 3, both Fluorescent Light-Emitting Polymer 1 and Phosphorescent Light-Emitting Polymer 1 have a similar peak emission wavelength at around 650 nm making it difficult to differentiate between these two polymers based on this peak wavelength alone (Fluorescent Light-Emitting Polymer 1 contains a peak at about 425 nm, however this is due to the large number of conjugated fluorene repeat units in the polymer and this peak would be smaller if not absent in a polymer optimised for efficient emission at about 650 nm, by inclusion of a larger quantity of the repeat unit derived from Monomer 2).

With reference to FIG. 4, the two polymers are clearly distinguishable from their time resolved photoluminescence spectra due to the much longer excited state lifetime of Phosphorescent Light-Emitting Polymer 1; the time taken for luminance of Fluorescent Light-Emitting Polymer 1 to decay to 1/1000 of its initial luminance is about 0.03 seconds; at this time, luminance of Phosphorescent Light-Emitting Polymer 1 has decayed to about ½ of its initial luminance. Accordingly, luminance detected after about 0.03 seconds following irradiation may be attributed to Phosphorescent Light-Emitting Polymer 1 with very little error arising from emission after this time from Fluorescent Light-Emitting Polymer 1.

1. A light-emitting composition comprising a first light-emitting marker and a second light-emitting marker wherein:
the first light-emitting marker comprises a first light-emitting material and a first binding group configured to bind to a first target analyte;
the second light-emitting marker comprises a second light-emitting material which is different from the first light-emitting material and a second binding group which is different from the first binding group and which is configured to bind to a second target analyte;
and wherein a luminescent lifetime of the first light-emitting material is shorter than a luminescent lifetime of the second light-emitting material.

2. The light-emitting composition according to claim 1 wherein the luminescent lifetime of the first light-emitting material is at least 10 times shorter than that of the second light-emitting material.

3. The light-emitting composition according to claim 1 wherein the first light-emitting material is selected from a fluorescent organic light-emitting material which does not comprise a metal complex and a fluorescent organic light-emitting material which comprises an aluminium metal complex.

4. The light-emitting composition according to claim 3 wherein the first light-emitting material is a first light-emitting polymer.

5. The light-emitting composition according to claim 4 wherein the light-emitting polymer is a conjugated light-emitting polymer.

6. The light-emitting composition according to claim 1 wherein the second light-emitting material is selected from an organic light-emitting material comprising a d-block or f-block metal complex.

7. The light-emitting composition according to claim 6 wherein the d-block or f-block metal complex is provided in an end-group, a repeat unit side-group or a repeat unit main chain group of a second light-emitting polymer.

8. The light-emitting composition according to claim 6 wherein the second light-emitting material is a phosphorescent iridium or platinum complex.

9. The light-emitting composition according to claim 1 wherein at least one of the first and second light-emitting markers is a light-emitting particle comprising a light-emitting particle core comprising the first or second light-emitting material and the first or second binding group bound to the light-emitting particle core.

10. The light-emitting composition according to claim 9 wherein the light-emitting particle core further comprises a matrix material.

11. The light-emitting composition according to claim 1 wherein at least one of the first and second light-emitting markers is dissolved in a solvent of the composition.

12. The light-emitting composition according to claim 1 wherein at least one of the first and second binding groups is a biomolecule.

13. The light-emitting composition according to claim 1 comprising one or more further light-emitting markers.

14. An assay method comprising contacting a sample with a light-emitting composition according to claim 1; irradiating the light-emitting markers with light of at least one peak wavelength; and measuring a luminance of any of the light-emitting markers bound to the respective target analytes after at least one predetermined time following the irradiation.

15. An assay method according to claim 14 wherein the first and second light-emitting materials both absorb light of a single peak wavelength of the irradiation light.

16. An assay method according to claim 14 wherein the first and second light-emitting materials absorb, and are irradiated with, light having different peak wavelengths.

17. An assay method according to claim 14 wherein the sample contacted with the light-emitting composition is analysed by flow cytometry.

18. An assay method according to claim 14 wherein an amount of the first target analyte and the second target analyte bound to the respective first and second light-emitting markers is determined.

19. An assay method according to claim 14 wherein the first and second target analytes are first and second cells.
